(19) United States
(12) Patent Application Publication
(10) Pub. No.: US 2013/0012766 A1
(43) Pub. Date: Jan. 10, 2013

(54) Sperm Delivery Device

(76) Inventor: Kim R Bayliss, Oakland, CA (US)

(21) Appl. No.: 13/179,532

(22) Filed: Jul. 10, 2011

(51) Int. Cl. A61B 17/43

(52) U.S. Cl. 600/35

(57) ABSTRACT

Sperm Delivery Device with a cup shaped sperm storage vessel, the side walls of the vessel having plurality of retaining receptacles, a resilient disk shaped portion integral with and parallel to the base of the sperm storage vessel, a thin closure membrane that dissolves at body temperature in a moist environment, and a closure ring having downwardly extending prongs that penetrate the retaining receptacles of said side walls of said sperm storage vessel that traps said closure membrane between the top of said sperm containing vessel and the underside of said closure ring. A preferred embodiment includes wherein said dissolvable closure sheet is made of gelatin or some other non-harmful dissolvable ingredient.

### BACKGROUND OF THE INVENTION

This invention relates generally to the field of human reproductive aids, and more particularly to a sperm delivery device.

Men have been impregnating women for the purpose of reproduction ever since men and women have existed. For reproduction to take place typically a man ejaculates sperm from his penis into the vaginal opening of a woman, the sperm travels the length of the vagina, through the uterus to reach an egg cell which has been released and is traveling down the fallopian tube to meet the sperm. The sperm fertilizes the egg and a new life begins to develop.

Although millions of sperm start the trip toward the awaiting egg, only a small fraction of the sperm actually make it all the way to the egg because they die along the way.

Women who have a hard time becoming pregnant have been seeking to increase the chance of sperm reaching and fertilizing an egg.

In some cases women who have a hard time getting pregnant go to a physician and have the physician inject the sperm into their uterus or vaginal opening so that the sperm is delivered to the entrance of the uterus sparing the sperm the tortuous trip down the vaginal canal and thereby increasing the number of viable sperms that reach the female egg. This procedure, although effective, is rather inconvenient and time consuming for the couple involved as well as being relatively expensive in that it involves a visit to a physician.

### SUMMARY OF THE INVENTION

The present invention relates to a sperm delivery device which is placed at the entrance of the cervix thereby positioning a greater number of sperm to enter the uterus, bypassing possible impediments in the vagina and or increasing the number of sperm successfully entering the uterus due to male factor infertility.

The Sperm Delivery Device comprising: a cup shaped vessel for the storage of sperm, which also serves as the base of the cup. The side walls of said vessel having plurality of retaining receptacles, a resilient disk shaped portion integral with and parallel to the base of said vessel for the storage of sperm.

A thin membrane that covers the sperm storage vessel that dissolves at body temperature in a moist environment. Membrane maybe impregnated with additives that enhance motility of sperm.

A resilient closure ring having downwardly extending prongs that penetrate the retaining receptacle of said side walls of said vessel for storage of sperm. The closure ring allows for thin membrane to be replaced and sperm delivery device to be reused more than once.

The sperm delivery device of the present invention is placed in the vagina while in a supine position, this allows for lip of the cervix to dip into the rim of the sperm delivery device. During ovulation a fertile quality cervical fluid exudes from the cervix which helps to transport sperm from the sperm delivery device up through the cervix and into the fallopian tubes thereby increasing chances that fertilization might occur.

The primary object of the invention is to provide a more economical way to deliver a male persons sperm to the entrance of the female persons uterus and to increase the chances of successful fertilization.

Other objects and advantages of the present invention will become apparent from the following descriptions and subsequent appended claims, taken in connection with the accompanying drawings. Wherein, by way of illustration and example, an embodiment of the present invention is disclosed.

### BRIEF DESCRIPTION OF THE DRAWINGS

FIG. 1 is an exploded view of the sperm delivery device of the present invention.

FIG. 2 is a side section of the sperm delivery device of the present invention.

### DETAILED DESCRIPTION OF THE PREFERRED EMBODIMENTS

Detailed descriptions of the preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

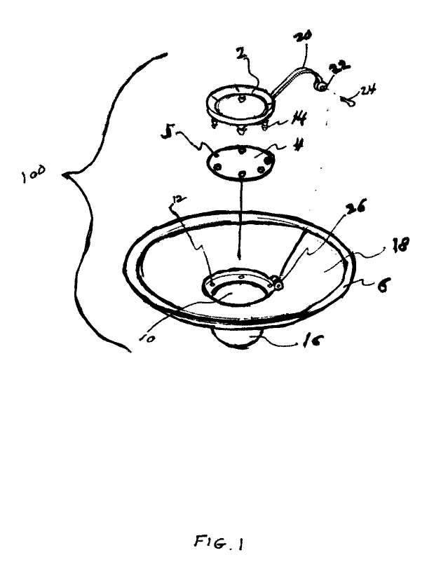

Referring now to FIG. 1 we see and exploded view of the sperm delivery device 100 of the present invention. Resilient disk 18 has a thicker integral ring 6 at its perimeter. A cup 16 is formed by side walls 10 mating with disk 18. The overall diameter of the disk 18 is approximately three inches in diameter and slightly concaved in shape. A thin, disk shaped membrane 4 acts as a top closure for cup 16. A ring 2 is the same diameter as the outer diameter of side wall 10. The ring 2 has plurality of prongs 14 extending down from the bottom surface of the ring. The prongs 14 penetrate through matching holes 5 in disk 4 and then penetrate into receptacles 12 located in the side wall 10 thereby removabley retaining membrane 4 onto the top surface of cup shape 16.

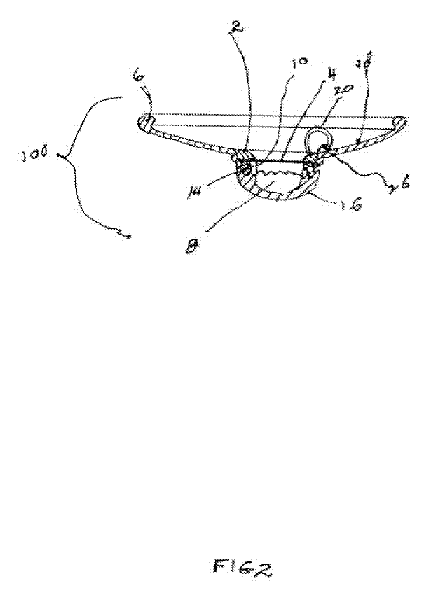

FIG. 2 shows a side section view of the sperm delivery device 100 of the present invention. Sperm 8 can be seen entrapped within side walls 10 and disk 18 at the bottom and disk 4 at the top. Disk 4 is made of a material that dissolves at body temperature in a moist environment. The preferred embodiment is a gelatin type material that has no harmful effects to the user when it dissolves within the vaginal canal. The material used would be similar to that used in a product called Vaginal Contraceptive Film manufactured by Apothicus Pharmaceutical Corporation of Oyter Bay, N.Y. This product is sold in square film sheets. The user inserts the sheet into the vagina and as close as possible to the opening of the uterus. The sheet dissolves due to body temperature and moisture thereby releasing contraceptive agent to reduce the chance of pregnancy. The film is made of a gelatin type substance and is impregnated with Nonoxynol-9, a contraceptive agent. For the purposes of the present invention the contraception agent is omitted leaving only a harmless, dissolvable membrane. The disk shaped portion 18 of the present invention and its integral perimeter ring 6 is similar in construction to a woman's diaphragm, is also commonly used as a contraceptive device. The disk 18 and ring 6 are typically made of resilient latex rubber material that can flex when inserted into the vaginal canal. However, the present invention may be made out of molded elastometric vinyl or other similar material. The closure ring 2 is preferably made of slightly stiffer material such as Monsanto's Santoprene or a similar injection moldable elastometric vinyl. The closure ring 2 is removable and replaceable so that a user can obtain a plurality of closure membranes 4 and reuse the device 100 numerous times. Closure ring 2 is attached to disk 18 by cord 20 at clevic point 26. The device 100 is inserted into the vagina in a similar way as a woman's diaphragm so that the closure membrane 4 dissolves the enclosed sperm 8 will be at the entry to the uterus and therefore have a shorter distance to travel and is exposed to a less hostile environment than with the normal intercourse method of impregnation. Because of the shorter distance there is a greater number of sperm available to fertilize the awaiting egg thereby increasing the chance of successful impregnation. The above described procedure can be done in the in the privacy of one's own home without the need of a physician's assistance thereby increasing the convenience and financial economy of the procedure.

While the invention has been described in connection with a preferred embodiment, it is not intended to limit the scope of the invention to the particular form set forth, but on the contrary, it is intended to cover such alternatives, modification, and equivalents as may be included within the spirit of the invention as defined by the appended claims.

What is claimed is:

1. A Sperm Delivery Device wherein said dissolvable closure sheet is made of gelatin, or some other non-harmful dissolving, ingredient.

2. A sperm Delivery Device as claimed in claim 1 wherein said device is reusable by addition of a new said closure membrane.

3. A cup shaped vessel for storage of sperm which also serves as the base of the cup, the side walls of said vessel having plurality of prong retaining receptacles. The sperm delivery device of the present invention is placed in the vagina and positioned over the cervix to transport sperm from the sperm delivery device up through the cervix and into the fallopian tubes thereby increasing chances that fertilization might occur.

4. A resilient concave disk shaped portion integral with and parallel to the base or cup of the sperm delivery device.

5. A thin closure membrane impregnated with motility inducing active ingredients that dissolves at body temperature and a moist environment increasing motility performance in sperm.

6. A closure ring having downwardly extending prongs that can penetrate said prong retaining receptacles of said side walls of said sperm storage vessel.

7. Thereby trapping said closure membrane between the top of said sperm containing vessel and the underside of said closure ring.

8. Wherein Sperm Delivery device accommodates animals with an ovulatory cycle and cervix.

9. Wherein Sperm Delivery Device accommodates humans with an ovulatory cycle and cervix.
